(19) United States
(12) Patent Application Publication
(10) Pub. No.: US 2018/0251721 A1
(43) Pub. Date: Sep. 6, 2018

(54) CELL CULTURE MODULE

(71) Applicants: COORSTEK KK, Tokyo (JP); SEKISHIN CO., LTD., Kyotango-shi (JP)

(72) Inventors: Shinya HASHIMOTO, Kanagawa (JP); Takafumi IMAIZUMI, Kanagawa (JP); Yukiko KIKUCHI, Kanagawa (JP); Kazunobu TSUNEMOTO, Kanagawa (JP); Takaaki ITOI, Kyoto (JP); Misao OBASE, Kyoto (JP)

(73) Assignees: COORSTEK KK; SEKISHIN CO., LTD.

(21) Appl. No.: 15/909,616

(22) Filed: Mar. 1, 2018

(30) Foreign Application Priority Data
Mar. 1, 2017 (JP) 2017-038200

(51) Int. Cl. C12M 1/34; C12M 1/36; C12M 1/00; C12M 3/04; C12M 3/06

(52) U.S. Cl. CPC C12M 41/34; C12M 1/36; C12M 1/005; C12N 5/0068; C12M 3/04; C12M 3/062; C12M 1/3415

(57) ABSTRACT

A cell culture module includes a cell culture substrate that has, on a surface thereof, a recessed portion that cultures a cell, in which at least a bottom surface of the recessed portion includes a porous body, a holding member that houses the cell culture substrate and that has an internal space partitioned into a first space and a second space by the cell culture substrate, and an oxygen sensor that is provided inside the first space of the holding member and that measures a concentration of dissolved oxygen in the culture medium. The oxygen sensor measures the concentration of dissolved oxygen in the vicinity of the cultured cell.

### TECHNICAL FIELD

The present invention relates to a culture module that cultures cell aggregates used for regenerative medicine and the like.

### BACKGROUND ART

In the related art, there is a perfusion culture apparatus as an apparatus that continuously cultures a cell, and various apparatuses are known as the perfusion culture apparatus.

For example, (1) a perfusion culture apparatus in which cells are two-dimensionally adhered to minute micro carriers and subjected to culture while stirring the micro carriers, (2) a radial flow bioreactor apparatus subjected to perfusion culture of floating cell aggregates, (3) a perfusion culture apparatus in which cell sheets are stacked, three-dimensionally formed and subjected to perfusion culture with a dedicated module are known. In addition, (4) a perfusion culture apparatus in which cells are adhered two-dimensionally to a substrate permeated with a culture medium and subjected to perfusion culture, and the like are known.

One of the present applicants has proposed (5) a porous ceramic culture substrate and a perfusion culture apparatus in which a culture medium supply device is connected to a module incorporating the porous ceramic culture substrate, as one of the perfusion culture apparatus of the above (4), in PTL 1.

In addition, in the perfusion culture apparatus of the above (3), a technique that controls a concentration of oxygen to produce a cell sheet in a short time is known as disclosed in PTL 2. Specifically, it is described that the concentration of carbon dioxide and oxygen is detected by a CO₂ and O₂ sensor connected to a culture vessel, and supply of oxygen or supply of carbon dioxide from a gas supply portion to the culture vessel is controlled.

### CITATION LIST

### Patent Literature

[PTL 1] JP 5666131 B2

[PTL 2] WO2013/002158 A1

### SUMMARY OF INVENTION

### Technical Problem

In the perfusion culture apparatus of the above (1) that cultures while stirring the micro carrier, and the radial flow bioreactor apparatus of the above (2), since the micro carrier and the cell aggregates are floated and cultured while stirring, there is an advantage that the components in the culture medium can be kept uniform.

However, there are problems that collisions between micro carriers or cell aggregates are likely to occur, that cells are likely to be stimulated by a flow of the culture medium, that it is difficult to control the size and shape of the cell aggregates, and that the loss of the cell is likely to increase, and the like.

In addition, in the perfusion culture apparatus of the above (3) in which the cell sheets are stacked, three-dimensionally formed and subjected to perfusion culture with the dedicated module, there are restrictions such as the necessity of technique and labor to prepare the cell sheets, limits on the number of overlapping cell sheets, and the size of the cell sheet that can be prepared at one time, so that in order to apply the apparatus to regenerative medicine which requires a large amount of cells, there is a problem of preparation efficiency.

Furthermore, in the perfusion culture apparatus of the above (4) in which the cells are adhered two-dimensionally to the substrate permeated with the culture medium and subjected to perfusion culture, and the porous ceramic culture substrate and the perfusion culture apparatus of the above (5) in which the culture medium supply device is connected to the module incorporating the porous ceramic culture substrate, there are advantages that there is no collision between cell aggregates and the size and shape of the cell aggregates are likely to be controlled as the floating culture described above, and that cell aggregates can be formed with relatively few processes in the case where a certain number of cells can be prepared.

However, in those perfusion culture apparatus, since the cell density was high, the perfusion rate was slow and stirring did not occur, an oxygen consumption rate of cell was faster than a natural diffusion rate of dissolved oxygen, a concentration gradient of oxygen concentration was generated in a culture space (in particular, the concentration gradient occurs remarkably as it approaches the cell aggregates), and there was a possibility that oxygen in the vicinity of the cell aggregates was insufficient.

The present inventors have intensively studied to solve the problem of controlling the concentration of oxygen in the vicinity of the cell on the premise of the cell culture substrate which can relatively easily form cell aggregates of a predetermined size, in which at least the bottom surface of the recessed portion is the porous body, and the perfusion culture apparatus in which the culture medium supply device is connected to the module incorporating the cell culture substrate, and have arrived at the present invention.

Furthermore, in PTL 2, although there is a technical disclosure of connecting a CO₂ and O₂ sensor to a culture vessel, there is no technical disclosure of the problem that a concentration gradient of oxygen concentration is generated in the culture space and the detection thereof.

The present invention is made under the above circumstances, and an object thereof is to provide a cell culture module capable of detecting the concentration of dissolved oxygen in a culture medium in the vicinity of the cell and controlling the concentration of dissolved oxygen in the vicinity of cultured cells.

### Solution to Problem

In order to achieve the above object, a cell culture module according to the present invention includes: a cell culture substrate that has, on a surface thereof, a recessed portion that cultures a cell, in which at least a bottom surface of the recessed portion includes a porous body; a holding member that houses the cell culture substrate and that has an internal space partitioned into a first space and a second space by the cell culture substrate; a supply port that is provided in the first space side of the holding member and that supplies a culture medium into the first space; an outlet port and an inlet port that are provided on the first space side of the holding member and that are for circulating the culture medium in the first space; a discharge port that is provided on the second space side of the holding member and that discharges the culture medium in the first space via the cell culture substrate; and an oxygen sensor that is provided inside the first space and that measures a concentration of dissolved oxygen in the culture medium in the first space.

As described above, in the cell culture module according to the present invention, the oxygen sensor that measures the concentration of dissolved oxygen in the culture medium in the first space is provided inside the first space of the holding member.

In accordance with a measurement value of the oxygen sensor, a negative pressure is applied (suction is performed) by the discharge port that discharges the culture medium, and the culture medium in the first space can be sucked via the cell culture substrate. By the suction, the culture medium having a low concentration of dissolved oxygen in the vicinity of the cultured cells is discharged from the recessed portion of the cell culture substrate, replaced with the culture medium having a high concentration of dissolved oxygen of a surface layer, and the oxygen deficiency of the cultured cell is eliminated.

In addition, the culture medium from the discharge port can be discharged and a new culture medium containing predetermined dissolved oxygen can be supplied from the supply port to allow the concentration of dissolved oxygen in the culture medium in the first space to be a predetermined concentration.

Furthermore, a control unit that controls the discharge rate and supply rate of the culture medium may be provided on the cell culture module and the discharge rate of the culture medium from the discharge port and the supply rate from the supply port may be automatically controlled according to the measurement value of the oxygen sensor to allow the concentration of dissolved oxygen in the culture medium inside the first space to be a predetermined concentration.

Furthermore, in accordance with the measurement value of the oxygen sensor, the culture medium may be led out from the outlet port and introduced again into the first space from the inlet port to circulate the culture medium. As described above, by circulating, a concentration gradient of dissolved oxygen in the culture medium inside the first space can be controlled.

In addition, in order to achieve the above object, a cell culture module according to the present invention includes: a cell culture substrate that has, on a surface thereof, a recessed portion that cultures a cell, in which at least a bottom surface of the recessed portion includes a porous body; a holding member that houses the cell culture substrate and that has an internal space partitioned into a first space and a second space by the cell culture substrate; a supply port that is provided in the second space side of the holding member and that supplies a culture medium into the first space via the cell culture substrate; an outlet port and an inlet port that are provided on the first space side of the holding member and that are for circulating the culture medium in the first space; a discharge port that is provided on the first space side of the holding member and that discharges the culture medium in the first space; and an oxygen sensor that is provided inside the first space and that measures a concentration of dissolved oxygen in the culture medium in the first space.

As described above, in the cell culture module according to the present invention, the oxygen sensor that measures the concentration of dissolved oxygen in the culture medium in the first space is provided inside the first space of the holding member.

In accordance with the measurement value of the oxygen sensor, the culture medium in the first space is discharged from the discharge port, and the culture medium is supplied from the second space side into the first space via the cell culture substrate. Therefore, the culture medium having a low concentration of dissolved oxygen in the vicinity of the cultured cell is discharged from the recessed portion of the cell culture substrate and replaced with a culture medium having a high concentration of dissolved oxygen to eliminate oxygen deficiency in the cultured cell.

Furthermore, in the cell culture module also, a control unit that controls the discharge rate and supply rate of the culture medium may be provided and the discharge rate of the culture medium from the discharge port and the supply rate from the supply port may be automatically controlled according to the measurement value of the oxygen sensor to allow the concentration of dissolved oxygen in the culture medium inside the first space to be a predetermined concentration. In addition, in accordance with the measurement value of the oxygen sensor, the culture medium may be led out from the outlet port and introduced again into the first space from the inlet port to circulate the culture medium. As described above, by circulating, the concentration gradient of dissolved oxygen in the culture medium inside the first space can be controlled.

Particularly, the oxygen sensor is preferably positioned at 0 mm or more and 5 mm or less from an upper surface of the cell culture substrate.

In the case where the oxygen sensor is positioned within the above range, the concentration of dissolved oxygen in the culture medium in the vicinity of the cell can be detected.

The oxygen sensor is preferably close to the upper surface of the cell culture substrate as much as possible (bringing into contact with the upper surface is not very preferable since there is a possibility that the sensor may be damaged due to impact at the time of contact), and exceeding 5 mm from the upper surface is not preferable because there is a possibility that the concentration of dissolved oxygen of the culture medium in the vicinity of the cell cannot be accurately measured under the influence of a concentration gradient of the oxygen concentration in the culture space.

Therefore, the oxygen sensor is preferably positioned at 0 mm or more and 5 mm or less from the upper surface of the cell culture substrate.

The position of the oxygen sensor indicates the distance from the lower end of the oxygen sensor. In addition, a disposition of the cell culture substrate and the oxygen sensor may be perpendicular or may be diagonal to the cell culture substrate. The oxygen sensor is more preferably disposed at a position perpendicular to the cell culture substrate because the concentration of dissolved oxygen in the vicinity of the cell can be accurately measured.

In addition, it is preferable to provide a liquid path connected to the discharge port that discharges the culture medium, and a suction portion that is connected to the liquid path and that applies a negative pressure to the inside of the second space of the holding member to apply the negative pressure to the culture medium inside the first space.

As described above, since the suction portion that applies the negative pressure to the inside of the second space is constituted, compared with the discharge by pressurization from inside the first space (above cell culture substrate), pressure on the cell can be reduced, so that damage to the cell can be suppressed.

The suction portion is not particularly limited, and is, for example, a syringe pump or a vacuum pump. Among these, in the case where the suction portion is constituted by the syringe pump, the negative pressure can be applied gently, to reduce the pressure applied to the cultured cell, and to replace the culture medium having a low concentration of dissolved oxygen in the vicinity of the cultured cell with the culture medium having a high concentration of dissolved oxygen of the surface layer while suppressing damage to the cell. As a result, the oxygen deficiency in the cultured cell is eliminated, which is more preferable.

### Advantageous Effects of Invention

According to the present invention, it is possible to obtain the cell culture module capable of detecting the concentration of dissolved oxygen in a culture medium in the vicinity of the cell and controlling the concentration of dissolved oxygen in the vicinity of the cultured cell.

### BRIEF DESCRIPTION OF DRAWINGS

FIG. 1 is a schematic view illustrating an embodiment of a cell culture module according to the present invention.

FIG. 2 is a view illustrating a cell culture substrate used in FIG. 1, and (a) of FIG. 2 is a perspective view, (b) of FIG. 2 is a plan view, and (c) of FIG. 2 is a cross-sectional view taken along line I-I of (b) of FIG. 2.

FIG. 3 is a plan view illustrating a holding member used in FIG. 1.

FIG. 4 is a cross-sectional view taken along line I-I of FIG. 3.

FIG. 5 is a cross-sectional view taken along line II-II of FIG. 3.

(a) of FIG. 6 is a box plot view showing an experimental result of Experiment 1, and (b) of FIG. 6 is an enlarged view of a portion where the distance of (a) of FIG. 6 is 5 mm, 7.5 mm and 10 mm.

FIG. 7 is a graph showing an experimental result of Experiment 2.

FIG. 8 is a schematic view illustrating an experimental apparatus of Experiment 3.

FIG. 9 is a graph showing a concentration of oxygen measured in an incubator by using the experimental apparatus illustrated in FIG. 8.

FIG. 10 is a graph showing a concentration of dissolved oxygen measured in the cell culture module by using the experimental apparatus illustrated in FIG. 8.

### DESCRIPTION OF EMBODIMENTS

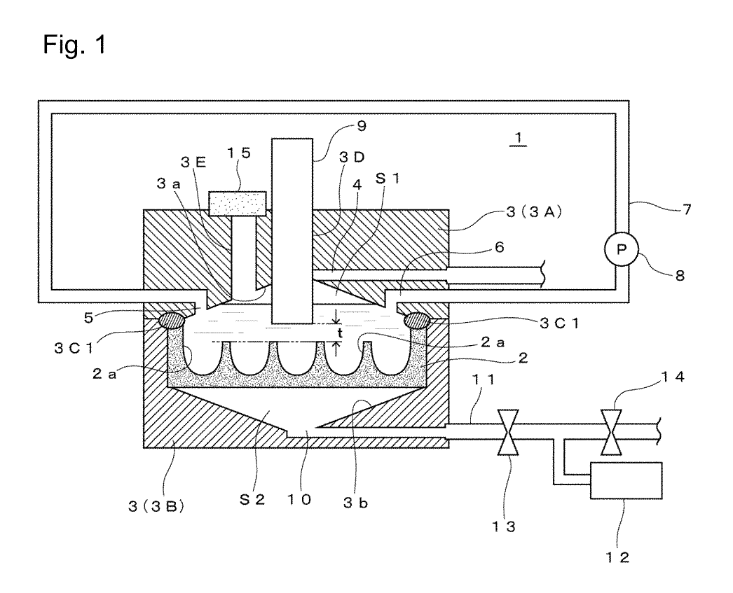

An embodiment of a cell culture module according to the present invention will be described based on FIGS. 1 to 5.

As illustrated in FIG. 1, the cell culture module 1 according to the present invention is provided with a cell culture substrate 2 that has, on the surface thereof, a recessed portion (well) 2a that cultures a cell, and in which at least a bottom surface of the recessed portion 2a includes a porous body.

In addition, a holding member 3 housing the cell culture substrate 2 and having an internal space partitioned into a first space S1 and a second space S2 by the cell culture substrate 2 is provided.

Furthermore, in order to measure the concentration of dissolved oxygen of the culture medium in the first space S1, an oxygen sensor 9 that measures the concentration of dissolved oxygen of the culture medium is provided inside the first space S1.

Furthermore, the concentration of dissolved oxygen in the culture medium is the concentration of oxygen dissolved in the culture medium, the concentration of saturated oxygen dissolved in a distilled water in an atmosphere at 37° C. and 1 atm (6.86 mg/L) is set to 20.9%, a state where oxygen is not completely dissolved (0 mg/L) is set to 0%, and other values are calculated by proportional calculation.

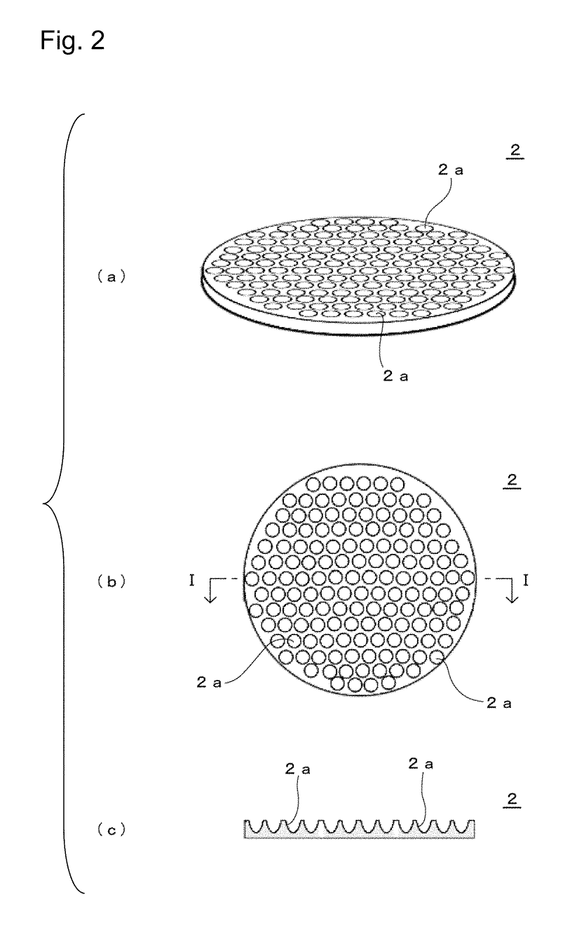

Furthermore, the cell culture substrate 2 will be described based on FIG. 2.

In the cell culture substrate 2, for example, a plurality of recessed portions (wells) 2a are formed on an upper surface thereof and bottom surfaces of the plurality of recessed portions (wells) 2a are formed to include porous bodies.

As described above, since at least the bottom surface of the recessed portion of the cell culture substrate 2 is formed to include the porous body, by sucking from a side opposite the upper surface of the cell culture substrate 2, a negative pressure can be applied to the culture medium in the first space S1.

As a result, it is possible to replace the culture medium in the vicinity of the cultured cell with the culture medium having a high concentration of dissolved oxygen in a surface layer in the first space S1. As a result, it is possible to eliminate oxygen deficiency in the cultured cell.

Furthermore, since at least the bottom surface of the recessed portion of the cell culture substrate 2 is formed to include the porous body, by sucking (applying negative pressure) the culture medium from the side opposite the upper surface of the cell culture substrate 2, the culture medium in the first space Si can be led out from the first space S1 into the second space S2.

The cell culture substrate 2 is not particularly limited as long as the bottom surface of the recessed portion is porous body, but specific examples of the porous body include a porous ceramic, a porous plastic and a porous organic membrane. Among these, the porous ceramic is preferable. For example, the cell culture substrate 2 can be formed by a ceramic porous sintered body having pores with an average pore diameter of 0.05 μm or more and 10 μm or less and a porosity of 25% or more and 50% or less, and the material include zirconia, yttria, titania, alumina, silica, alumina-silica, hydroxyapatite, and β-tricalcium phosphate, which have high biological safety. Among these, alumina, zirconia, hydroxyapatite, β-tricalcium phosphate, and titania, which are confirmed to have the biological safety are more preferable. Particularly, zirconia or alumina is preferable.

The cell culture substrate 2 is a porous sintered body prepared by using the above-described material, and thus can sufficiently supply the culture medium to the entire cell aggregate adhering to the culture substrate.

Next, the holding member and the oxygen sensor will be described based on FIG. 1 and FIGS. 3 to 5.

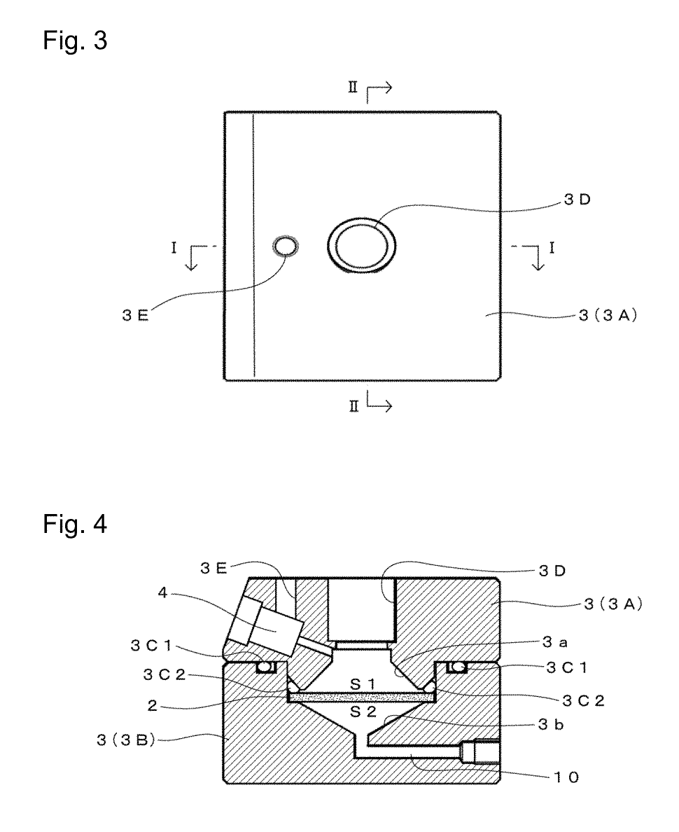

FIG. 1 illustrates a schematic configuration, and a specific configuration of the holding member 3 is illustrated in FIGS. 3 to 5.

As illustrated in FIG. 1, the holding member 3 is provided with an upper member 3A constituted by plastic and having a recessed portion 3a formed on the lower surface and a lower member 3B constituted by plastic and having a recessed portion 3b formed on the upper surface.

The space formed by the recessed portion 3b of the lower member 3B houses the cell culture substrate 2, and is brought into contact with the upper member 3A having the recessed portion 3a formed on the lower surface. As a result, the holding member 3 in which the internal space is partitioned into a first space S1 and a second space S2 by the cell culture substrate 2 is formed.

Furthermore, an O-ring 3C1 is provided at a fitting portion between the upper member 3A and the lower member 3B, and air tightness is maintained. In addition, as illustrated in FIG. 4, an O-ring 3C2 is provided between the upper member 3A and the cell culture substrate 2 and the air tightness is maintained.

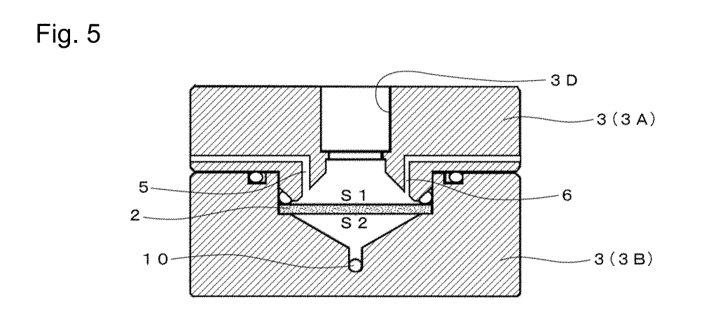

In addition, as illustrated in FIGS. 4 and 5, the upper member 3A forming the first space S1 is provided with a supply port 4 that supplies the culture medium into the first space S1, an outlet port 5 and an inlet port 6 for circulating the culture medium in the first space S1.

As illustrated in FIG. 1, the outlet port 5 and the inlet port 6 are connected by a liquid path 7, and are configured so that the culture medium in the first space S1 can be circulated by a pump 8.

Furthermore, a through-hole 3D communicating with the recessed portion 3a is formed in the upper central part of the upper member 3A of the holding member 3, and is configured so that the oxygen sensor 9 can be attached thereto.

The oxygen sensor 9 is one that measures the concentration of dissolved oxygen of the culture medium in the first space S1, and is disposed so that the distance dimension t between the lower surface of the oxygen sensor 9 and the upper surface of the cell culture substrate 2 is 0 mm or more and 5 mm or less.

In this manner, since the oxygen sensor is disposed close to the cell culture substrate 2, the concentration of dissolved oxygen in the culture medium in the vicinity of the cell can be measured.

In addition, the oxygen sensor 9 is preferably close to the upper surface of the cell culture substrate 2 as much as possible. Furthermore, in the case of bringing the oxygen sensor 9 into contact with the upper surface, since there is a possibility of sensor damage, it is preferable to be gentle contact.

On the other hand, in the case where the distance dimension t exceeds 5 mm from the upper surface of the cell culture substrate 2, there is a possibility that the concentration of dissolved oxygen of the culture medium in the vicinity of the cell cannot be accurately measured under the influence of a concentration gradient of the concentration of oxygen in the culture space.

Therefore, the oxygen sensor is preferably positioned at 0 mm or more and 5 mm or less from the upper surface of the cell culture substrate.

In addition, an air hole 3E communicating with the first space S1 is formed in an upper portion of the upper member 3A of the holding member 3. It is configured so that air can be supplied into the first space S1 via the air hole 3E.

In addition, a filter 15 is provided in the air hole 3E, so that the air flowing in from the air hole 3E is prevented from being contaminated with bacteria, viruses and the like. Although a case where the air hole 3E is illustrated is described, it may not be provided in the case where air is supplied into the first space S1 by other constitutions (liquid path or the like).

Furthermore, a discharge port 10 that discharges the culture medium is provided in the lower member 3B forming the second space S2 of the holding member 3, and a liquid path 11 is connected to the discharge port 10. In addition, the liquid path 11 is provided with a suction portion 12 that sucks (applies a negative pressure) the culture medium inside the first space S1 of the holding member 3.

In addition, reference numerals 13 and 14 in FIG. 1 are switching valves. Although not illustrated in FIG. 1, a discharge pump is provided at the rear stage of a switching valve 14, and is configured so that the suction portion 12 and the discharge pump can be switched.

Subsequently, the operation and action of the cell culture module according to the above embodiment will be described based on FIG. 1.

During cell culture, the concentration of dissolved oxygen of the culture medium in the first space S1 is measured by the oxygen sensor 9. As a result, in the case where dissolved oxygen is within a predetermined concentration range, that state is maintained.

Thereafter, as a result of the measurement by the oxygen sensor 9, in the case where the concentration is lower than the predetermined concentration, the suction portion 12 is operated to apply a negative pressure to the second space S2, and the culture medium in the first space is sucked via the cell culture substrate 2.

By this suction, the culture medium having a low concentration of dissolved oxygen in the vicinity of the cultured cell is discharged from the inside of the recessed portion of the cell culture substrate 2, replaced with the culture medium having a higher concentration of dissolved oxygen in the surface layer, and the oxygen deficiency in the cultured cell is eliminated.

In addition, as described above, since the suction portion 12 that applies a negative pressure to the inside of the second space is constituted, compared with the discharge by pressurization from inside the first space (above cell culture substrate), pressure on the cell can be reduced, so that damage to the cell can be suppressed.

In addition, in the cell culture module 1, since the outlet port 5 and the inlet port 6 for circulating the culture medium in the first space S1 are provided, in accordance with the measurement result by the oxygen sensor 9, the pump 8 may be operated to circulate the culture medium in the first space S1.

Furthermore, in accordance with the result of the measurement by the oxygen sensor 9, the switching valves 13 and 14 may switch from the suction portion 12 to the discharge pump, and the culture medium from the discharge port 10 may be discharged.

Here, a new culture medium having a predetermined concentration of dissolved oxygen is supplied into the first space Si from the supply port 4 connected to a culture medium supply device (not illustrated), so that the concentration of dissolved oxygen of the culture medium is maintained within the predetermined concentration range.

In addition, the cell culture module 1 may be provided with a control unit that controls the discharge rate and the supply rate of the culture medium, and the discharge rate of the culture medium from the discharge port and the supply rate from the supply port may be automatically controlled according to the measurement value of the oxygen sensor to allow the concentration of dissolved oxygen in the culture medium inside the first space to be a predetermined concentration.

In addition, as a modified example of the above embodiment, a supply port may be provided on a second space side of a holding member to supply culture medium into a first space via a cell culture substrate, and a discharge port may be provided on a first space side of the holding member to discharge the culture medium in the first space. Furthermore, similar to the above embodiment, an oxygen sensor that measures the concentration of dissolved oxygen of the culture medium in the first space is provided inside the first space.

Even in the modified example, in accordance with the measurement value of the oxygen sensor, the culture medium in the first space is discharged from the discharge port, and the culture medium is supplied from the second space side into the first space via the cell culture substrate. Therefore, the culture medium having a low concentration of dissolved oxygen in the vicinity of the cultured cell is discharged from the recessed portion of the cell culture substrate and replaced with a culture medium having a high concentration of dissolved oxygen to eliminate oxygen deficiency in the cultured cell.

In addition, even in the modified example, similar to the above embodiment, a control unit that controls the discharge rate and supply rate of the culture medium may be provided and the discharge rate of the culture medium from the discharge port and the supply rate from the supply port may be automatically controlled according to the measurement value of the oxygen sensor to allow the concentration of dissolved oxygen in the culture medium inside the first space to be a predetermined concentration. In addition, in accordance with the measurement value of the oxygen sensor, the culture medium may be led out from the outlet port and introduced again into the first space from the inlet port to circulate the culture medium. As described above, by circulating, the concentration gradient of dissolved oxygen in the culture medium inside the first space can be controlled.

In the above modified example, the supply port is provided on the second space side of the holding member, the culture medium is supplied into the first space via the cell culture substrate, the discharge port is provided on the first space side of the holding member, and the culture medium in the first space is discharged. Furthermore, similar to the above embodiment, a discharge port and a suction portion may be provided on the second space side of the holding member.

As explained above, the cell culture module according to the present invention is a cell culture module housing the cell culture substrate in which at least the bottom surface of the recessed portion includes the porous body, a culture medium supply device or the like is connected to the cell culture module, and the dissolved oxygen deficiency can be eliminated in the culture medium in the vicinity of the cell in the culture apparatus performing the perfusion culture. Since the oxygen sensor is provided in the cell culture module according to the present invention, the discharge and supply rate of the culture medium can be controlled and the concentration of dissolved oxygen in the vicinity of the cell can be maintained constant according to the measurement value of the sensor.

More specifically, since the oxygen sensor of the cell culture module according to the present invention is positioned at 0 mm or more and 5 mm or less from the upper surface of the cell culture substrate, the concentration of dissolved oxygen in the culture medium in the vicinity of the cell can be accurately measured.

In addition, in the case where the suction portion that applies a negative pressure to inside the second space is provided, since it is possible to reduce the pressure on the cell, compared with the discharge by pressurization from inside the first space (above cell culture substrate), it is possible to replace the culture medium having a low concentration of dissolved oxygen in the vicinity of the cultured cell with the culture medium having a higher concentration of dissolved oxygen of the surface layer while suppressing damage to the cell.

### EXAMPLES

Hereinafter, although the present invention will be described more specifically based on Examples, the present invention is not limited by the following Examples.

### Experiment 1

By using the cell culture module illustrated in FIG. 1, an experiment was performed on the concentration gradient of the concentration of dissolved oxygen in the culture space.

As a cell culture substrate, a cell culture substrate which is a porous ceramic sintered body having pores with an average pore diameter of 0.14 μm and a porosity of 48% and is a cell culture substrate with a thickness of 3 mm and a diameter of 34 mm, in which 3,000 recessed portions (wells) with a depth of 0.5 mm and a diameter of 0.5 mm are formed, was used.

A suspension (2×10⁶ cells/ml) in which 5×10⁶ human iPS cells were suspended in 2.5 ml of DMEM medium containing 10% fetal bovine serum (FBS) was seeded on the cell culture substrate.

In addition, the culture medium was supplied so as to have a depth of 15 mm from the upper surface of the cell culture substrate. The oxygen sensor was provided while changing the position from the upper surface of the cell culture substrate to 0 mm, 1 mm, 2.5 mm, 5 mm, 7.5 mm, and 10 mm, and the oxygen concentration in the culture medium at each position was measured.

The results are shown in FIG. 6. (a) of FIG. 6 is a box plot view plotting the concentration of dissolved oxygen (%) on the vertical axis and the distance from the cell culture substrate to the oxygen sensor on the horizontal axis. (b) of FIG. 6 is an enlarged view of a portion where the distances are 5 mm, 7.5 mm and 10 mm.

As shown in (a) FIG. 6, it was confirmed that in the case where the oxygen sensor is provided at a distance of 5 mm or less from the upper surface of the cell culture substrate (within a region of 35% of the depth of the culture medium in the first space), the concentration of dissolved oxygen rapidly decreased. In addition, as shown in (b) of FIG. 6, in the case where the oxygen sensor is provided at a distance exceeding 5 mm from the upper surface of the cell culture substrate (within the region of 35% of the depth of the culture medium), the measurement result varied greatly and the concentration of oxygen could not be measured accurately.

Therefore, it is preferable that the oxygen sensor is positioned (disposed) at 0 mm or more and 5 mm or less from the upper surface of the cell culture substrate.

### Experiment 2

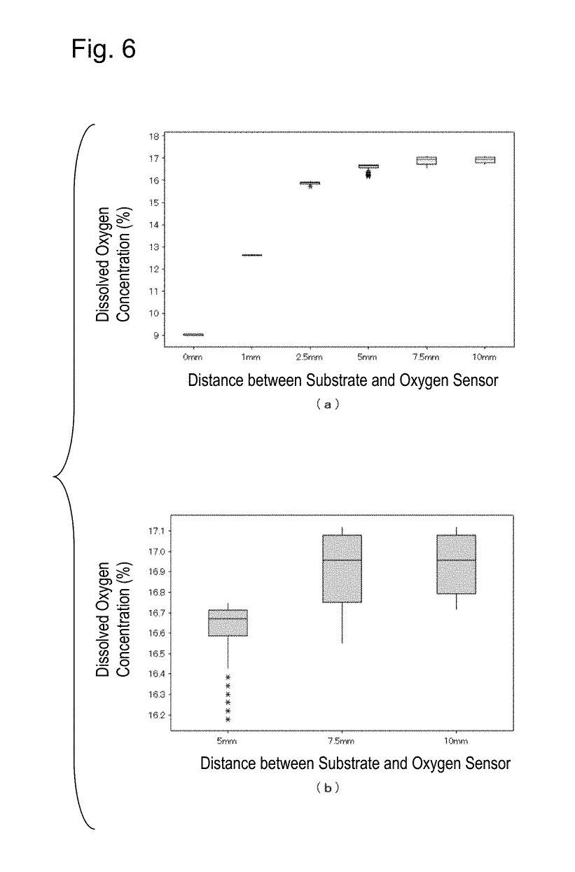

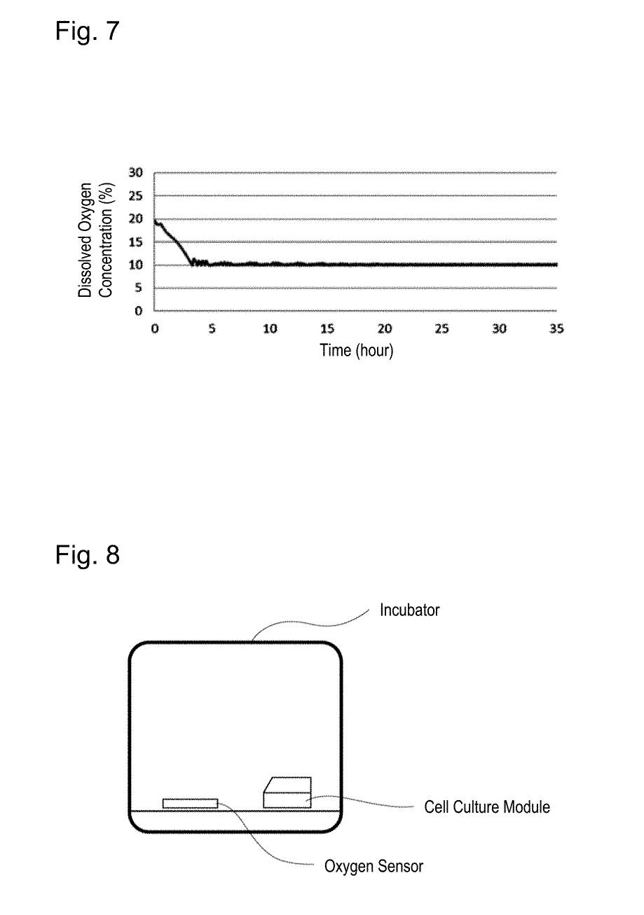

Under the same conditions as in Experiment 1, the oxygen sensor was positioned at 1 mm from the upper surface of the cell culture substrate and the oxygen concentration was controlled to 10%. Specifically, in the case where the oxygen concentration is 10% or less by oxygen consumption of the cell, the culture medium was circulated at a flow rate of 0.3 ml/min, and the oxygen concentration was maintained at 10%. The results are shown in FIG. 7.

As shown in FIG. 7, it is understood that the concentration of dissolved oxygen in the vicinity of the cell is maintained at approximately 10%. That is, it was confirmed that the concentration of dissolved oxygen in the vicinity of cell can be controlled by circulating the culture medium.

### Experiment 3

As illustrated in FIG. 8, a cell culture module and an oxygen sensor were disposed in an incubator, and the concentration of oxygen in the incubator was measured while culturing the cell in the cell culture module.

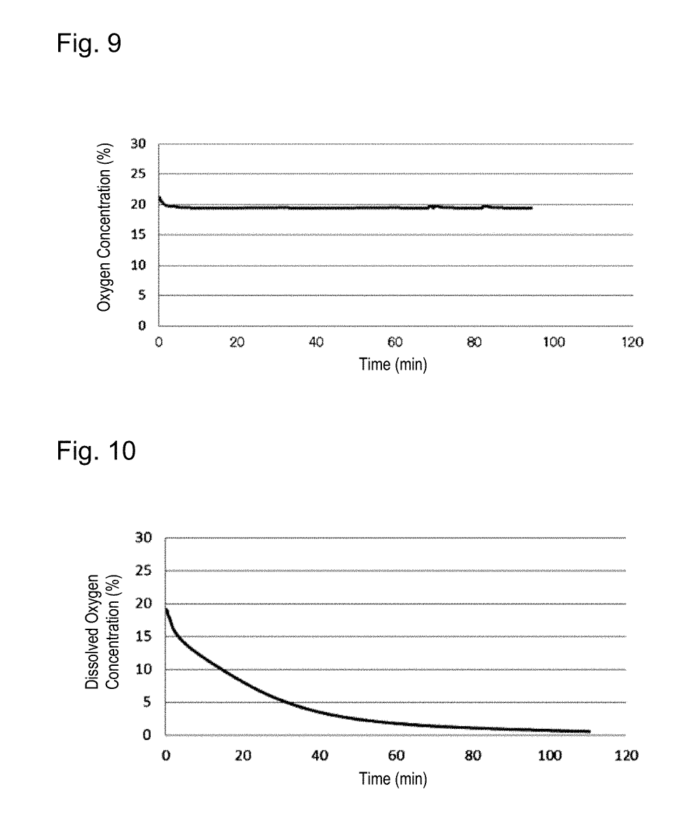

In this experiment, a cell culture substrate which is a porous ceramic sintered body having pores with an average pore diameter of 0.14 μm and a porosity of 48% and is a cell culture substrate with a thickness of 3 mm and a diameter of 34 mm, in which 3,000 recessed portions (wells) with a depth of 0.5 mm and a diameter of 0.5 mm are formed, was used. A suspension (2×10⁶ cells/ml) in which 5×10⁶ human mesenchymal stem cells were suspended in 2.5 ml of DMEM medium containing 10% fetal bovine serum (FBS) was seeded on the cell culture substrate. The measurement result is shown in FIG. 9.

In addition, an experiment was performed under the same conditions as in the above experiment except that the oxygen sensor was positioned 1 mm from the upper surface of the cell culture substrate. That is, the same suspension (2×10⁶ cells/ml) was seeded using the same cell culture substrate, and the concentration of oxygen at 1 mm from the upper surface of the cell culture substrate was measured. The result is shown in FIG. 10.

As can be seen from FIGS. 9 and 10, it was found that the oxygen concentration in the incubator differs from the oxygen concentration in the module.

Therefore, it was found that even if the oxygen sensor is disposed in the incubator and the change in the oxygen concentration in the incubator is measured, the measurement value is different from the concentration of dissolved oxygen in the vicinity of the cultured cell, so that oxygen deficiency in cultured cell could not be detected.

### REFERENCE SIGNS LIST

1 cell culture module

2 cell culture substrate

3 holding member

3A upper member

3B lower member

3D through-hole

3E air hole

3a recessed portion of upper member

3b recessed portion of lower member

4 supply port

5 outlet port

6 inlet port

7 liquid path

8 pump

9 oxygen sensor

10 discharge port

11 liquid path

12 suction portion

13 switching valve

14 switching valve

15 filter

S1 first space

S2 second space

t distance dimension between the oxygen sensor (lower surface) and the upper surface of the cell culture substrate

1. A cell culture module comprising:
a cell culture substrate that has, on a surface thereof, a recessed portion that cultures a cell, wherein at least a bottom surface of the recessed portion comprises a porous body;
a holding member that houses the cell culture substrate and that has an internal space partitioned into a first space and a second space by the cell culture substrate;
a supply port that is provided in the first space side of the holding member and that supplies a culture medium into the first space;
an outlet port and an inlet port that are provided on the first space side of the holding member and that are for circulating the culture medium in the first space;
a discharge port that is provided on the second space side of the holding member and that discharges the culture medium in the first space via the cell culture substrate; and
an oxygen sensor that is provided inside the first space and that measures a concentration of dissolved oxygen in the culture medium in the first space.

2. A cell culture module comprising:
a cell culture substrate that has, on a surface thereof, a recessed portion that cultures a cell, wherein at least a bottom surface of the recessed portion comprises a porous body;
a holding member that houses the cell culture substrate and that has an internal space partitioned into a first space and a second space by the cell culture substrate;
a supply port that is provided in the second space side of the holding member and that supplies a culture medium into the first space via the cell culture substrate;
an outlet port and an inlet port that are provided on the first space side of the holding member and that are for circulating the culture medium in the first space;
a discharge port that is provided on the first space side of the holding member and that discharges the culture medium in the first space; and
an oxygen sensor that is provided inside the first space and that measures a concentration of dissolved oxygen in the culture medium in the first space.

3. The cell culture module according to claim 1, wherein the oxygen sensor is positioned at 0 mm or more and 5 mm or less from an upper surface of the cell culture substrate.

4. The cell culture module according to claim 1, wherein the oxygen sensor is positioned within a region of 35% of a depth of the culture medium in the first space.

5. The cell culture module according to claim 1, further comprising:
a liquid path connected to the discharge port that discharges the culture medium; and
a suction portion that is connected to the liquid path and that applies a negative pressure to the inside of the second space of the holding member to apply the negative pressure to the culture medium inside the first space.

6. The cell culture module according to claim 2, wherein the oxygen sensor is positioned at 0 mm or more and 5 mm or less from an upper surface of the cell culture substrate.

7. The cell culture module according to claim 2, wherein the oxygen sensor is positioned within a region of 35% of a depth of the culture medium in the first space.

8. The cell culture module according to claim 3, further comprising:
a liquid path connected to the discharge port that discharges the culture medium; and
a suction portion that is connected to the liquid path and that applies a negative pressure to the inside of the second space of the holding member to apply the negative pressure to the culture medium inside the first space.

9. The cell culture module according to claim 4, further comprising:
a liquid path connected to the discharge port that discharges the culture medium; and
a suction portion that is connected to the liquid path and that applies a negative pressure to the inside of the second space of the holding member to apply the negative pressure to the culture medium inside the first space.
